(19) United States
(12) Patent Application Publication
(10) Pub. No.: US 2025/0312268 A1
(43) Pub. Date: Oct. 9, 2025

(54) COMPOSITIONS AND METHODS FOR TREATING SKIN PROBIOTIC DEFICIENCY SYNDROME

(71) Applicant: AllerPops Corporation, Los Alamos, NM (US)

(72) Inventor: Cliff Shunsheng Han, Los Alamos, NM (US)

(73) Assignee: AllerPops Corporation, Los Alamos, NM (US)

(21) Appl. No.: 19/172,794

(22) Filed: Apr. 8, 2025

Related U.S. Application Data
(60) Provisional application No. 63/575,935, filed on Apr. 8, 2024.

(51) Int. Cl. A61K 9/00; A61K 36/47; A61K 36/889; A61K 45/06

(52) U.S. Cl. CPC A61K 9/0014; A61K 36/47; A61K 36/889; A61K 45/06

(57) ABSTRACT

A method and composition of promoting a desired topical microbiota in a subject to treat skin probiotic deficiency syndrome comprising providing a topical composition comprising a primary oil comprising castor oil; a secondary oil comprising palm oil; a tertiary oil comprising coconut oil; a texture enhancement agent comprising beeswax; and wherein the composition is applied to affected areas once daily for treatment of a skin probiotic deficiency syndrome to increase dermatological expression of short-chain fatty acid producing bacteria.

### CROSS-REFERENCE TO RELATED APPLICATIONS

This application claims the benefit of priority of U.S. Provisional Patent Application Ser. No. 63/575,935, filed on Apr. 8, 2024, and titled “COMPOSITIONS AND METHODS FOR TREATING SKIN PROBIOTIC DEFICIENCY SYNDROME” which is incorporated by reference herein in its entirety.

### FIELD OF THE INVENTION

The present invention generally relates to the field of skin probiotics. In particular, the present invention is directed to compositions and methods for treating skin probiotic deficiency syndrome.

### BACKGROUND

Skin probiotic deficiency syndrome includes inflammatory diseases caused by a hypersensitive immune system, such as eczema, arthritis, urticaria, joint/muscle pains and (skin) autoimmune diseases.

### SUMMARY OF THE DISCLOSURE

In an aspect, a composition for treating skin probiotic deficiency syndrome comprising a primary oil comprising castor oil; a secondary oil comprising palm oil; a tertiary oil comprising coconut oil; a texture enhancement agent and wherein the composition is suitable to increase dermatological expression of short-chain fatty acid producing bacteria for treating a skin probiotic deficiency syndrome.

In yet another non-limiting aspect, a method of promoting a desired topical microbiota in a subject to treat skin probiotic deficiency syndrome comprising: providing a topical composition comprising a primary oil comprising castor oil; a secondary oil comprising palm oil; a tertiary oil comprising coconut oil; a texture enhancement agent comprising beeswax; and wherein the composition is applied to affected areas once daily for treatment of a skin probiotic deficiency syndrome to increase dermatological expression of short-chain fatty acid producing bacteria.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram illustrating an exemplary embodiment of a composition for treating skin probiotic deficiency syndrome; and

FIG. 2 is a block diagram illustrating an exemplary embodiment of a method of promoting a desired topical microbiota in a subject to treat skin probiotic deficiency syndrome.

The drawings are not necessarily to scale and may be illustrated by phantom lines, diagrammatic representations and fragmentary views. In certain instances, details that are not necessary for an understanding of the embodiments or that render other details difficult to perceive may have been omitted.

These and other aspects and features of non-limiting embodiments of the present invention will become apparent to those skilled in the art upon review of the following description of specific non-limiting embodiments of the invention in conjunction with the accompanying drawings.

### DETAILED DESCRIPTION

Described herein are compositions and methods for treating Skin Probiotic Deficiency Syndrome (SPDS). A composition may include coconut oil and castor oil. A composition may be applied body with focus to neck, armpits, groins, knees, elbows and affected areas once daily for treatment, or after each shower for maintenance and prevention. Other ingredients may be used to dilute and prevent side effects. Palm oil may be used in place of coconut oil. A composition may be used as a treatment and/or prophylaxis for SPDS.

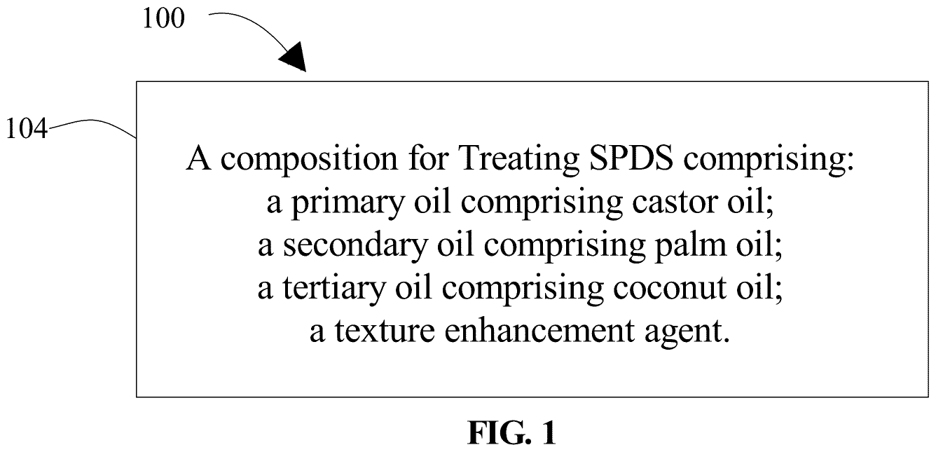

Referring now to FIG. 1, an exemplary embodiment of composition 100 is described. Composition 100 for treating skin probiotic deficiency syndrome comprises a primary oil comprising castor oil. Castor oil may be extracted from the seeds of the castor bean plant Ricinus Communis. Castor oil may contain ricinoleic acid which may contain antimicrobial properties and provide skin barrier support. Castor oil may aid in modulating skin inflammation and supporting microbial colonization. In an embodiment, castor oil may be present at a weight percent level of from about greater than 0.1 weight percent to about 1.0 weight percent. In an embodiment, castor oil may be present at a weight percent level of from about greater than 0.1 weight percent to about 99.9 weight percent. In an embodiment, primary oil may include but is not limited to Jamaican black castor oil; argan oil; olive oil; peppermint oil; jojoba oil; sweet almond oil; shea butter; and/or tamanu oil. In an embodiment, primary oil may be present at a weight percent level of from about greater than 0.1 weight percent to about 99.9 weight percent.

With continued reference to FIG. 1, composition 100 comprises a secondary oil comprising palm oil. Palm oil may be extracted from the fruit of the palm oil tree such as Elaeis guineensis. Palm oil may provide palmitic acid and tocotrienols for skin barrier repair and antioxidant support. In an embodiment, palm oil may be present at a weight percent level of from about greater than 0.01 weight percent to about 0.1 weight percent. In an embodiment, palm oil may be present at a weight percent of from about greater than 0.1 weight percent to about 99.9 weight percent. In an embodiment, secondary oil may include but is not limited to shea butter, cocoa butter, and/or babassu oil. In an embodiment, secondary oil may be present at a weight percent level of from about greater than 0.1 weight percent to about 99.9 weight percent.

With continued reference to FIG. 1, composition 100 comprises a tertiary oil comprising coconut oil. Coconut oil may be extracted from the coconut palm tree, Cocos nucifera. Coconut oil may modulate microbial populations and act as an emollient. In an embodiment, coconut oil may be present at a weight percent of from about greater than 40 weight percent to about 70 weight percent. In an embodiment, coconut oil may be present at a weight percent of from about greater than greater than 0.1 weight percent to about 99.9 weight percent. In an embodiment, tertiary oil may include but is not limited to mango utter, palm kernel oil, avocado oil, almond oil, jojoba oil, and the like. In an embodiment, tertiary oil may be present at a weight percent level of from about greater than 0.1 weight percent to about 99.9 weight percent.

With continued reference to FIG. 1, composition 100 comprises a texture enhancement agent. A “texture enhancement agent,” as used in this disclosure, is an ingredient that improves physical properties of composition 100 when applied to skin. A texture enhancement agent may affect how composition 100 feels on a subject, how composition 100 spreads on a subject, consistency of composition 100, texture of composition 100, thickness of composition 100 and the like. Texture enhancement agent may provide structure and occlusive properties. Texture enhancement agent may enhance residence time of primary, secondary, and tertiary oils on the skin and aid in supporting microbial growth and colonization. Texture enhancement agent may include but is not limited to cetyl alcohol, stearic acid, beeswax, silicones such as dimethicone, gums such as xanthan gum, guar gum, emulsifying wax, shea butter, cocoa butter, carbomer, and/or lanolin. Texture enhancement agent may include beeswax present at a weight percent level of from about greater than about 0.01 weight percent to about 0.1 weight percent. In an embodiment, beeswax may be present at a weight percent of from about greater than 0.1 weight percent to about 99.9 weight percent. In an embodiment, texture enhancement agent may be present at a weight percent level of from about greater than 0.1 weight percent to about 99.9 weight percent.

With continued reference to FIG. 1, composition 100 may be comprised of 500 grams of a primary oil comprising castor oil; 50 grams of a secondary oil comprising palm oil; 950 grams of a tertiary oil comprising coconut oil; and 50 grams of a texture enhancement agent comprising beeswax.

With continued reference to FIG. 1, composition 100 may increase expression of short-chain fatty acid producing bacteria found on the skin that may be decreased due to factors such as excessive skin hygiene, antibiotic use, deodorant use, and/or genetics. Composition 100 may increase expression of short-chain fatty acid found within skin microbiota. The skin microbiota may be comprised of one or more microbes that keep skin barrier strong and help prevent infection. Composition 100 may increase expression of short-chain fatty acid producing bacteria within skin microbiota including Cutibacterium Acnes. Composition 100 may increase expression of other short-chain fatty acid producing bacteria within skin microbiota including but not limited to Staphylococcus Epidermidis, Corynebacterium species, Bifidobacterium Longum, and the like. Promotion of short-chain fatty acid producing bacteria within skin microbiota may also inhibit fungus and other opportunistic skin pathogens.

With continued reference to FIG. 1, composition 100 may be formulated into any topical delivery mechanism, including but not limited to a balm, a cream, an ointment, a gel, a lotion, a solution, a spray, a foam, and/or a patch. Composition 100 may contain one or more penetration enhancement agents to improve absorption including but not limited to liposomes, niosomes, microemulsions, nano emulsions, solid lipid nanoparticles, transfersomes, and/or micelles.

With continued reference to FIG. 1, composition 100 may be delivered using enhanced delivery mechanisms including but not limited to microneedles, iontophoresis, sonophoresis, electroporation, thermal ablation, and/or jet injection.

Skin probiotics are short-chain fatty acid (SCFA) producing bacteria, such as Cutibacterium Acnes. They calm the immune system with SCFAs leading to a mutually beneficial, peaceful condition. Skin probiotic deficiency, the reduction of the good bacteria, leads to insufficient SCFAs, resulting hypersensitive immune system related to skin, joints, and muscles.

With continued reference to FIG. 1, the major causes of skin probiotic deficiency syndrome includes excessive skin hygiene and antibiotics. Excessive skin hygiene is the primary cause in modern society that contributed to the boom of skin allergies and autoimmune diseases. The primary biological niche for skin probiotics are hair facile and sebaceous glands. Too much skin hygiene, soapy shower and antibacterial detergents, suppress the beneficial bacteria. On the other hand, too much of these bacteria may cause skin infection. Therefore, we propose moderate skin hygiene, avoiding extreme or poor skin hygiene. Antibiotics/deodorant use may suppress the skin probiotics and change the community structure that prevents the good bacteria from coming back properly. Another cause may include genetics. The inherit immune strength may moderate the relationships between skin probiotics and the host.

With continued reference to FIG. 1, the symptoms of skin probiotic deficiency syndrome may include any combination of eczema, urticaria, joint/muscle pains and (skin) autoimmune diseases, which include prostate enlargement. Diagnosis of skin probiotic deficiency syndrome (SPDS) may include tests that help to distinguish skin probiotic deficiency syndrome from other skin infections. Treatment of SPDS contains correction of skin probiotic deficiency and of gut and oral probiotic deficiency if they exist. The correction of skin probiotic deficiency includes two coordinated efforts. Feed the probiotics with skin prebiotic supplements. They are usually oily creams. Apply the skin prebiotic cream to armpits, groin and affected areas daily if you have any irritation on the skin, in any joints and muscles. You can reduce the frequency of use for maintenance. Application of skin prebiotic supplements to scalp and neck may be beneficial to skin and hair. Pause application of these products if you have infection in related areas. Stop excessive skin hygiene. Remove antibacterial skin care products. Reduce shower frequency to once or twice a week with gentle shampoo.

With continued reference to FIG. 1, SPDS can be prevented by helping skin probiotics recover after cure of infections, such as flu, Covid, pneumonia, after antibiotic usage, and after giving birth. In addition, have a moderate skin hygiene routine and applying skin prebiotic cream regularly as in the treatment section. The calm and peaceful immune system will stop/slow the development of new sensitivity against pollen, food or us. The outcome of existing conditions depends on the specific situations.

1. Allergic skin issues can be well controlled with minimal/no symptoms.

2. Joint/muscle pains may disappear completely.

3. Autoimmune diseases. The symptoms can be reduced to manageable.

With continued reference to FIG. 1, in some embodiments, a composition for treating and/or preventing SPDS may include a post-probiotic. In some embodiments, a method of treating and/or preventing SPDS may include administration of a post-probiotic. A post-probiotic may include a short chain fatty acid. In some embodiments, administration of a composition including a post-probiotic may lead to instant relief of one or more symptoms of SPDS. In some embodiments, a post-probiotic may include acetate, propionate, butyrate, isobutyrate, valerate, and/or isovalerate. In some embodiments, a composition may include a compound and/or organism capable of producing a post-probiotic. For example, a composition may include a bacterium capable of producing a post-probiotic. In some embodiments, a composition including a post-probiotic and/or a bacterium capable of producing a post-probiotic may be administered to the skin. In some embodiments, a composition including a post-probiotic and/or a bacterium capable of producing a post-probiotic may be administered such that the post-probiotic is made available in the gut. In some embodiments, a first composition including coconut oil and/or castor oil is administered to the skin and a second composition including a post-probiotic and/or a bacterium capable of producing a post-probiotic may be administered such that the post-probiotic is made available in the gut. In some embodiments, administration to the gut may include oral administration. In some embodiments, a post-probiotic may mitigate inflammation by modulating production of cytokines by immune cells.

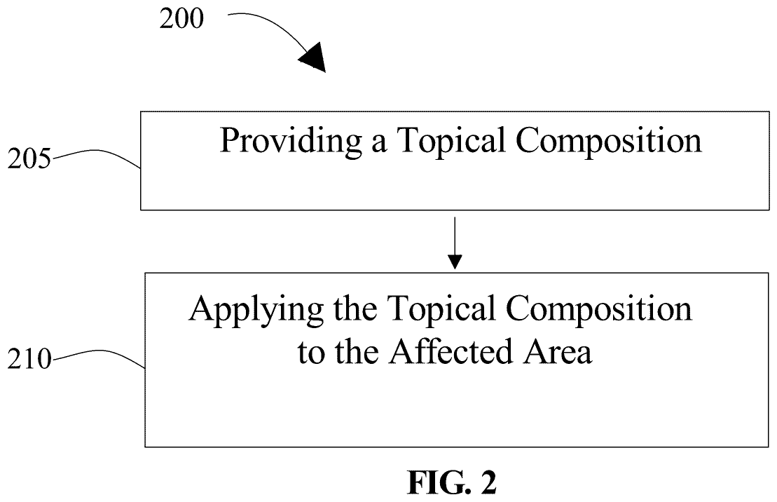

Referring now to FIG. 2, an exemplary embodiment 200 of a method of promoting a desired topical microbiota in a subject to treat skin probiotic deficiency syndrome is illustrated. At step 205, a topical composition comprising a primary oil comprising castor oil; a secondary oil comprising palm oil; a tertiary oil comprising coconut oil; and a texture enhancement agent comprising beeswax is provided. Topical composition may include any composition as described above in more detail in reference to FIG. 1.

With continued reference to FIG. 2, at step 210 composition is applied to affected areas once daily for treatment of a skin probiotic deficiency syndrome to increase dermatological expression of short-chain fatty acid producing bacteria. The composition may be applied after each show for maintenance of skin microbiota. The composition may be applied after each shower for prevention of recurrence of SPDS. The composition may be applied after each shower for primary prevent of SPDS. The composition may be applied to one or more armpits. The composition may be applied to a groin. The composition may be applied to a joint. The composition may be applied to a muscle. The composition may aid in treating symptoms associated with SPDS such as urticaria, autoimmune disease, allergic skin issues, joint pain, muscle pain, eczema, and/or any other hypersensitive skin condition.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve compositions and methods according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A composition for treating skin probiotic deficiency syndrome comprising:
a primary oil comprising castor oil;
a secondary oil comprising palm oil;
a tertiary oil comprising coconut oil;
a texture enhancement agent and
wherein the composition is suitable to increase dermatological expression of short-chain fatty acid producing bacteria for treating a skin probiotic deficiency syndrome.

2. The composition of claim 1, wherein the skin probiotic deficiency syndrome further comprises arthritis.

3. The composition of claim 1, wherein the skin probiotic deficiency syndrome further comprises urticaria.

4. The composition of claim 1, wherein the skin probiotic deficiency syndrome further comprises joint or muscle pain.

5. The composition of claim 1, wherein the skin probiotic deficiency syndrome further comprises autoimmune disease.

6. The composition of claim 1, wherein the short-chain fatty acid producing bacteria further comprises Cutibacterium Acnes.

7. The composition of claim 1, wherein the primary oil is present at a weight percent level of from about greater than about 0.1 wt. % to about 1.0 wt. %.

8. The composition of claim 1, wherein the secondary oil is present at a weight percent level of from about greater than about 0.01 wt. % to about 0.1 wt. %.

9. The composition of claim 1, wherein the tertiary oil is present at a weight percent level of from about greater than about 40 wt. % to about 70 wt. %.

10. The composition of claim 1, wherein the texture enhancement agent further comprises beeswax present at a weight percent level of from about greater than about 0.01 wt., % to about 0.1 wt. %.

11. A method of promoting a desired topical microbiota in a subject to treat skin probiotic deficiency syndrome comprising:
providing a topical composition comprising:
a primary oil comprising castor oil;
a secondary oil comprising palm oil;
a tertiary oil comprising coconut oil;
a texture enhancement agent; and
wherein the topical composition is applied to affected areas once daily for treatment of a skin probiotic deficiency syndrome to increase dermatological expression of short-chain fatty acid producing bacteria.

12. The method of claim 11, wherein the topical composition is applied after each shower for maintenance.

13. The method of claim 11, wherein the topical composition is applied after each shower for prevention.

14. The method of claim 11, wherein the affected area further comprises an armpit.

15. The method of claim 11, wherein the affected area further comprises a groin.

16. The method of claim 11, wherein the texture enhancement agent further comprises beeswax.

17. The method of claim 11, wherein the short-chain fatty acid producing bacteria further comprises Cutibacterium Acnes.

18. The method of claim 11, wherein the short-chain fatty acid producing bacteria further comprises Bifidobacterium longum.

19. The method of claim 11, wherein the skin probiotic deficiency syndrome further comprises arthritis.

20. The method of claim 11, wherein the skin probiotic deficiency syndrome further comprises autoimmune disease.
